(19) United States
(12) Patent Application Publication
(10) Pub. No.: US 2005/0281890 A1
(43) Pub. Date: Dec. 22, 2005

(54) Methods and compositions for wound healing

(76) Inventor: Chandan K. San, New Albany, OH (US)

Correspondence Address: CALFEE HALTER & GRISWOLD, LLP, 800 SUPERIOR AVENUE, SUITE 1400, CLEVELAND, OH, 44114, US

(21) Appl. No.: 10/871,158

(22) Filed: Jun. 18, 2004

(51) Int. Cl. A61K 33/40

(52) U.S. Cl. 424/616

(57) ABSTRACT

Methods, devices, and compositions for treating lesions in animals, comprising hydrogen peroxide in concentrations that are less than conventionally used. The methods, devices, and compositions provide an increased rate of wound healing.

### DESCRIPTION OF THE INVENTION

1. Field of the Invention

This invention generally relates to methods and compositions for treating wounds.

2. Background of the Invention

For years it has been believed that reactive oxygen species (ROS) are primarily damaging to living cells. In fact, ROS such as hydrogen peroxide and ozone have been used for their powerful oxidizing effects as disinfectants. These oxidizing effects are non-specific; in addition to destroying unwanted microorganisms, considerable collateral damage is produced. Thus, in the doses traditionally used in disinfecting, hydrogen peroxide is destructive to living tissue.

However, it has been discovered that at lower doses, hydrogen peroxide has surprising effects on the healing process. The present invention relates to the use of low-dose hydrogen peroxide for its wound healing effects.

### SUMMARY OF THE INVENTION

The present invention provides methods of increasing the rate of lesion healing in mammals comprising applying to the lesion about 500 nanomoles to about 50 micromoles of hydrogen peroxide per square centimeter of lesion. In some embodiments, the methods comprise applying about 1 to about 50, or about 1 to about 10, or about 1 to about 2 micromoles of hydrogen peroxide per square centimeter of lesion. The hydrogen peroxide can be applied to the lesion in a source chosen from, for example, enzymatic and chemical sources. In some embodiments, the source of hydrogen peroxide is chemical, and the source is hydrogen peroxide.

The invention also provides methods of increasing the rate of lesion healing in a mammal comprising applying hydrogen peroxide to the lesion at a rate of about 500 nanomoles to about 50 micromoles of hydrogen peroxide per square centimeter of lesion over a period of from about 12 hours to about 24 hours. In some embodiments, the hydrogen peroxide is applied at a rate of from about 1 to about 10 micromoles. The hydrogen peroxide can be applied in a pharmaceutically acceptable composition, which may be in a form chosen from, for example, gels, lotions, ointments, creams, pastes, and liquids. The hydrogen peroxide can be applied in a pharmaceutically acceptable device, including but not limited to, bandages, surgical dressings, gauzes, adhesive strips, surgical staples, clips, hemostats, intrauterine devices, sutures, trocars, catheters, tubes, and implants. Implant include, but are not limited to, pills, pellets, rods, wafers, discs, and tablets.

The device can comprise a polymeric material, which can comprise an absorbable material. In some embodiments, the absorbable material comprises a synthetic material. Synthetic materials can be chosen from cellulosic polymers, glycolic acid polymers, methacrylate polymers, ethylene vinyl acetate polymers, ethylene vinyl alcohol copolymers, polycaptrolactam, polyacetate, copolymers of lactide and glycolide, polydioxanone, polyglactin, poliglecaprone, polyglyconate, polygluconate, and combinations thereof. In some embodiments, the absorbable material comprises a non-synthetic material. Non-synthetic material can be chosen from catgut, cargile membrane, fascia lata, gelatin, collagen, and combinations thereof.

The device can comprise a polymeric material, which can comprise a nonabsorbable material. In some embodiments, the nonabsorbable material comprises a synthetic material. Synthetic materials can be chosen from nylons, rayons, polyesters, polyolefins, and combinations thereof. In some embodiments, the nonabsorbable material comprises a non-synthetic material. Non-synthetic materials can be chosen from silk, dermal silk, cotton, linen, and combinations thereof.

The method can be used to treat lesions chosen from wounds, ulcers, and burns. Wounds can be chosen from acute wounds and chronic wounds. The wounds can be chosen from full thickness wounds and partial thickness wounds. Acute wounds can be chosen from, for example, surgical wounds, penetrating wounds, avulsion injury, crushing injury, shearing injury, burn injury, laceration, and bite wound. Chronic wounds can be chosen from, for example, arterial ulcers, venous ulcers, pressure ulcers, and diabetic ulcers.

The present invention also provides a hydrogen peroxide delivery device for administration to a lesion, comprising hydrogen peroxide and a carrier material, which device releases said hydrogen peroxide for a period of time which is at least about 12 hours, wherein the hydrogen peroxide released from the device is in insufficient concentration to produce necrosis of the lesion. In some embodiments, the device releases from about 0.5 to 50 μmol hydrogen peroxide/cm² wound/12 hr to about 0.5 to 50 μmol hydrogen peroxide/cm² wound/24 hr. The carrier material can comprise a polymeric material. In some embodiments, the polymeric material comprises an absorbable material. In some embodiments, the polymeric material comprises a synthetic material.

The invention also provides a composition for treating lesions in mammals comprising: hydrogen peroxide and a pharmaceutically acceptable carrier, wherein a unit dose of the composition comprises from about 0.5 to about 50 μmol hydrogen peroxide/cm² wound. In some embodiments, the carrier comprises a gel material, and in some embodiments, the carrier comprises a liquid material.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

FIG. 1. Presence of ROS at wound-site. A. H₂O₂ concentration in wound fluid. Hunt/Schilling wire mesh cylinders were subcutaneously implanted on the back of 5 week old C57BL/6 mice via incisional wounding. After 5d, the wound fluid was collected and the steady-state H₂O₂ concentration in the fluid was measured using a real-time electrochemical technique as described herein below. The baseline was collected in PBS. Wound fluid (0.15 ml) was added to DPBS (1 ml) at time indicated with an arrow. Using a standard curve, the concentration of H₂O₂ determined in the wound fluid was 1.1 μM. B. EPR spectra of DMPO adduct measured from wound rinsate. The spectra were acquired from DMPO (100 mM, 0.1 ml) effluents collected from wound cavity at 0 h (sham control, upper panel) 12 h post-wounding (lower panel). The spectra in lower panel was identified as that of DMPO—OH with the following coupling constants: aN=14.90 G, aH=14.90. The data acquisition parameters were: microwave frequency, 9.8682 GHz; sweep width, 100 G; microwave power, 20 mW; modulation amplitude, 0.5 G; modulation frequency, 100 kHz; time constant 80 msec. C. Superoxide production in normal skin and wound edge tissue. The wound edge samples were harvested at 12 h after wounding and immediately frozen in OCT. Fresh 30-micron sections were incubated with DHE (0.01 mM, 20 min, 200x,) to detect O₂⁻ and visualized by confocal microscopy.

FIG. 2. Catalase over-expression attenuates healing. The skin to be wounded was subcutaneously injected once with either catalase and LacZ (control) adenoviruses (10¹¹ pfu) 5 days before wounding to allow for maximum over-expression of catalase at the wound site. Two 8×16 mm full-thickness secondary intention wound were placed on the dorsal skin of 8 wk old C57BL/6 mice (FIG. 2). A. Western blot of infected skin showing catalase over-expression in the side treated with AdCatalase (AdCat) virus compared to the side treated with Control Ad LacZ virus. Blots were reprobed with β-actin to show equal loading of samples. B. Wound closures are shown as percentage of area of initial wound determined on the indicated day after wounding. Dotted line represents standard healing curve of saline treated C57BL/6 mice (open circles, ◯) without viral infection. AdCat Treatment (closed triangles,▾); AdlacZ treatment (closed circles, ●)); *p<0.05, compared to LacZ treated side. C. Masson trichrome staining was performed on formalin fixed paraffin sections of regenerated skin at the wound-site sampled on the day both wounds closed. AdCat side shows broader HE region indicative of incomplete (vs. control) regeneration of skin, consistent with slower closure. Es, eschar; G, granulation tissue; HE.

FIG. 3. Topical H₂O₂ on wound closure: dosage a key issue. Two 8×16 mm full-thickness excisional wounds (inset) were placed on the dorsal skin of C57BL/6 male mice (8 wk old). Each of the two wounds was topically treated either with H₂O₂ or saline A. Low-dose of H₂O₂ (1.25 micromoles/wound; or 0.025 ml of 0.15% solution/wound; once daily, days 0-4, open circles, ◯) treatment facilitated closure moderately compared to placebo treated (closed circles, ●) side. *, p<0.05. B. Low dose H₂O₂ treatment is not toxic to wound microflora. For determination of surface microflora, wounds (treated with either 1.25 micromole H₂O₂/wound, open bar, or saline, closed bar) were swabbed (24-48 h post wounding) for 20 sec with an alginate-tipped applicator. Quantitative assessment of surface bacterial load was performed. For deep tissue wound microflora, 48 h after wounding eschar tissue was removed, wound bed tissue underneath eschar was sampled and quantitative assessment of bacterial load was performed. Values shown represent mean±SD of CFU of four observations. C. High dose (high, 25 micromoles/wound; closed circles, ●, 0.025 ml of 3% solution versus low, 1.25 micromoles/wound or 0.025 ml of 0.15%; open circles, ◯, once daily days 0-4, of H₂O₂ adversely affected closure. *, p<0.05; compared to low dose H₂O₂ treatment. A higher concentration (inset) of H₂O₂ (62.5 micromoles/wound, left side treated; 0.025 ml of 7.5% solution/wound, once on day 0) treatment causes necrotic tissue damage and severe injury leading to death of mice.

FIG. 4. Wound and H₂O₂-induced changes in angiogenesis related genes, vascularization and wound-edge blood flow. Paired excisional wounds (FIG. 2) were either treated with placebo saline or H₂O₂ (1.25 micromole/wound, days0-4, once daily). Wound-edge tissue was collected at indicated times after wounding. A. Ribonuclease protection assay (RPA) showing kinetics of angiogenesis-related mRNA expression in a placebo-treated wound. B. Low dose H₂O₂ treatment (1.25 micromole/wound, once daily, days 0-4) to wounds further augmented wound-induced Flt-1 and VEGF mRNA expressions as determined using RPA. C. Blood flow imaging of wounds was performed non-invasively using a Laser Doppler blood flow imaging device. Images reflecting the blood flow (right panel) and a digital photo (region of interest; left panel) from post-heal tissue are shown. Data i.e., mean±SD of the blood flow is presented (bar graph). The mean represents the arithmetic mean of all valid blood flow values for pixels within the region of interest. The results show that the treatment resulted in increased blood flow, a functional outcome of enhanced angiogenesis. D. Day 8 post-wounding, wound-edge was cryosectioned and vascularization was estimated by staining for CD31 (red, rhodamine) and DAPI (blue, nuclei); higher abundance of CD31 red stain in section obtained from H₂O₂ treated side (bottom) reflect better vascularization vs control (top).

FIG. 5. H₂O₂-induced phosphorylation of focal adhesion kinase (FAK) in microvascular endothelial cells and wound edge tissue. Human microvascular endothelial cells (HMEC-1) were treated with H₂O₂ for indicated dose and duration. Phosphorylation of FAK was detected using Western blot and phosphorylation site-specific antibodies against FAK. Native FAK or β-actin was blotted to show equal loading. A. Effect of various dose of H₂O₂ treatment on phosphorylation (Ty 925) state of FAK. B. Kinetics of site-specific activation phophorylation of FAK in HMEC cells following H₂O₂ (0.1 mM) treatment. C. Paired excisional wounds (FIG. 2) were either treated with placebo saline or H₂O₂ (1.25 micromole/wound). Wound-edge tissue was collected 30 min after wounding. FAK phosphorylation in wound edge tissue was determined using Western blot. Data from three animals (#1-#3) are shown.

FIG. 6. MCP-1 and p47phox deficiency impairs dermal healing. Two excisional wounds (FIG. 2) were-laced on the dorsal skin of 8 wk old C57BL/6, MCP-1 or p47phox KO mice. Each of the two wounds was treated with either saline or H₂O₂ (1.25 micromoles/wound; days 0-4). A. RPA showing kinetics of monocyte/macrophage chemotactic protein related mRNA expression in placebo-treated wounds of wild-type (C57BL/6) mice. B. Wound closures in saline (closed circles, ●) treated wounds of C57BL/6 and H₂O₂ (closed triangles,▾) or saline (open circles, ◯) treated MCP-1 KO mice are shown as percentage of area of initial wound. * p<0.05; compared to C57BL/6 saline treatment. #, p<0.05; compared to KO saline treatment. C. Wound closures in saline (closed circles, ●) treated wounds of C57BL/6 and H₂O₂ (closed triangles,▾) or saline (open circles, ◯) or p47 Phox KO mice are shown as percentage of area of initial wound. *p<0.05; compared to C57BL/6 saline treatment. #, p<0.05; compared to KO saline treatment. D. Keratin 14 (green fluorescence) expression in skin of p47phox KO mice harvested from wound sites after closure on day 18 post-wounding. Note higher expression of keratin 14 in control side compared to H₂O₂-treated side indicating healing is ongoing on the control side, while H₂O₂ treated side shows keratin 14 expression comparable to normal skin.

### DESCRIPTION OF THE EMBODIMENTS

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are incorporated by reference in their entirety.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

The present invention generally relates to the use of hydrogen peroxide or its sources in lesion healing. In some embodiments, the rate of healing is increased, and in some embodiments, there is a reduction in scarring. The invention can generally be used to treat any damage to a living body in which the body's natural repair process will occur. The invention can be used to treat lesions in animals, such as mammals, and specifically including humans.

The term “lesion” is used herein in its generic sense, meaning that it encompasses all sorts of wounds and injuries. “Wound” can also be used in its generic sense, meaning that it encompasses wounds, burns, ulcers, etc. “Wound” and “lesion” may be used interchangably herein, and unless the context specifically dictates otherwise, no distinction is intended. Lesions can be wounds, burns, ulcers, etc. Lesions/wounds can be acute or chronic. Wounds can be full thickness, i.e., penetrating all layers of skin, or partial thickness, i.e., penetrating less than all layers of skin. Examples of acute wounds include, but are not limited to, surgical wounds, penetrating wounds, avulsion injuries, crushing injuries, shearing injuries, burn injuries, lacerations, and bite wounds. Examples of chronic wounds include, but are not limited to, ulcers, such as arterial ulcers, venous ulcers, pressure ulcers, and diabetic ulcers. Of course, acute wounds can become chronic wounds.

The composition that is applied to the lesion to be treated contains hydrogen peroxide, or a source of hydrogen peroxide. The concentration of hydrogen peroxide applied to the lesion is less than that amount that is conventionally used, and in some embodiments is less than that amount that produces an oxidizing effect on microbes or other living cells, and in some embodiments is less than that amount that produces a necrotic effect on contacted tissue. In some embodiments, the amount of hydrogen peroxide applied to a lesion is from about 500 nanomoles (nmol) to about 50 micromoles (μmol) per square centimeter (cm²) of lesion. In some embodiments, the amount of hydrogen peroxide applied to a lesion is from about 5 μmol to about 500 μmol per cubic centimeter (cm³) of lesion.

The amount of hydrogen peroxide applied to a lesion can range from about 500, 600, 700, 800, or 900 nmol, or 1, 2, 3, 4, 5, 6, 7, 8, 9, or about 10 μmol or higher, to about 2, 3, 4, 5, 6, 7, 8, 9,10, 15, 20, 25, 30, 40, or about 50 μmol, per square centimeter (cm²) of lesion. The amount can range, for example, from 1-50, 1-25, 1-10, or 1-2 μmol, per square centimeter (cm²) of lesion. The amount of hydrogen peroxide applied to a lesion can range from about 5, 6, 7, 8, 9, 10, 20, 30, 40, 50, 60, 70, 80, 90, or about 100 μmol or higher, to about 20, 30,40, 50, 60, 70, 80, 90, 100, 150, 200, 250, 300, 400, or about 500 μmol, per cubic centimeter (cm³) of lesion. The amount can range, for example, from 10-500, 10-250, 10-100, or 10-20 μmol, per cubic centimeter (cm³) of lesion.

The concentration of hydrogen peroxide applied to a lesion can range from about 10, 15, 20, 25, 30, 35, 40, 45, 50, or about 75 mM to about 50, 55, 60, 65, 70, 75, 80, 85, 90, 95, or 100 mM, or higher. Thus, the concentration of hydrogen peroxide can range from about 10 to about 100 mM, or from about 25 to about 75 mM, or from about 40 to about 60 mM.

The hydrogen peroxide can be applied in any form of vehicle or carrier, including but not limited to, liquids, gels, lotions, creams, pastes, and ointments. The means of application will depend upon what form the hydrogen peroxide takes: liquids can be sprayed or poured, for example; gels, lotions, creams, pastes, and ointments can be rubbed or massaged, for example. These and other forms, and/or carriers/vehicles, for hydrogen peroxide delivery, are described in publications such as Remington's Pharmaceutical Science, and other similar publications.

The delivery forms can be homogeneous, e.g., forms in which the hydrogen peroxide is in solution, or heterogeneous, e.g., forms in which hydrogen peroxide is contained within liposomes or microspheres. The forms can produce an immediate effect, and can alternatively, or additionally, produce an extended effect. For example, liposomes, or microspheres, or other similar means of providing an extended release of hydrogen peroxide, can be used to extend the period during which the hydrogen peroxide is exposed to the lesion; non-encapsulated hydrogen peroxide can also be provided for an immediate effect.

The delivery forms can also take the form of devices, which can deliver hydrogen peroxide to a lesion for a desired period of time. Devices include, but are not limited to, bandages, surgical dressings, gauzes, adhesive strips, surgical staples, clips, hemostats, intrauterine devices, sutures, trocars, catheters, tubes, and implants. Implants include, but are not limited to, pills, pellets, rods, wafers, discs, and tablets.

Devices according to the invention can be prepared according to known methods, and can include, or be made from, polymeric material. In some instances, the polymeric material will be an absorbable material and in other instances, a non-absorbable material. Devices can, of course, include both absorbable and non-absorbable materials.

Absorbable materials can be synthetic materials and non-synthetic materials. Absorbable synthetic materials include, but are not limited to, cellulosic polymers, glycolic acid polymers, methacrylate polymers, ethylene vinyl acetate polymers, ethylene vinyl alcohol copolymers, polycaptrolactam, polyacetate, copolymers of lactide and glycolide, polydioxanone, polyglactin, poliglecaprone, polyglyconate, polygluconate, and combinations thereof. Absorbable non-synthetic materials include, but are not limited to, catgut, cargile membrane, fascia lata, gelatin, collagen, and combinations thereof.

Nonabsorbable synthetic materials include, but are not limited to nylons, rayons, polyesters, polyolefins, and combinations thereof. Non-absorbable non-synthetic materials include, but are not limited to, silk, dermal silk, cotton, linen, and combinations thereof.

Combinations of the foregoing devices and carriers/vehicles are also envisioned. For example, a hydrogen peroxide gel or ointment can be impregnated into a bandage or wound dressing for delivery of the hydrogen peroxide to the desired location. As another example, an implantable absorbable device can be loaded with a hydrogen peroxide solution and release the solution from the device over a period as desired. The physical form used to deliver the hydrogen peroxide is not critical and the choice or design of such devices is well within the level of skill of one in the art.

Hydrogen peroxide can be delivered to the desired target site as hydrogen peroxide, per se, or it can be delivered as a precursor. For example, superoxide is transformed into hydrogen peroxide by superoxide dismutase, which is naturally present in animals. Thus, hydrogen peroxide can be delivered to a target site by administering superoxide, which is transformed into hydrogen peroxide. Hydrogen peroxide-like peroxides can be delivered by delivering, for example, tert-butyl hydroperoxide. All of these types of sources can be considered chemical sources of hydrogen peroxide.

Hydrogen peroxide also is formed naturally in the body by a reaction between hemoglobin and oxygen to produce superoxide, which is then converted to hydrogen peroxide by superoxide dismutase. The hydrogen peroxide is naturally degraded in the body by an enzyme called catalase. Hydrogen peroxide can be allowed to accumulate at a lesion site by administering a catalase inhibitor to the target site. Hydrogen peroxide can also be caused to accumulate by administering additional superoxide dismutase. Delivery of hydrogen peroxide to a lesion site in this manner is considered to have been performed by enzymatic source. This can also be considered a natural source of hydrogen peroxide, as opposed to an extraneous source.

Hydrogen peroxide can also be generated as a byproduct of a number of reactions, including, for example: 1) glucose+glucose oxidase; 2) xanthine+xanthine oxidase; 3) hypoxanthine+xanthine oxidase; and 4) ascorbate+ascorbate oxidase. Hydrogen peroxide concentration can also increased in a body by the overexpression of rac1 and rac2, NADPH oxidase, and superoxide dismutase. All of these are considered enzymatic sources of hydrogen peroxide and are within the scope of the invention.

The hydrogen peroxide is delivered to the desired target site at least once. In some embodiments the hydrogen peroxide is delivered to the target site two, three, four, five, six, seven, eight, nine, ten, or more times. The delivery can be as often as every two, four, six, eight, ten, twelve, fourteen, sixteen, eighteen, twenty, twenty-two, or twenty-four hours, or more. Where repeated doses are desired, devices or other carriers can be “programmed” to release doses of hydrogen peroxide at desired times. For example, a microsphere formulation can include unencapsulated hydrogen peroxide for an immediate effect on administration; an encapsulated component that delivers a second dose at twenty-four hours; and an encapsulated component that delivers a third dose at forty-eight hours. The treatment strategy is left to the practitioner; the design of devices or carriers, etc., is within the level of skill in the art.

It may be desirable to provide for other conditions in the practice of the present invention. For example, it may be desirable to ensure that the target region of the lesion is sufficiently oxygenated; generally, it is sufficient that atmospheric oxygen be present. It also may be desirable to maintain a desired level of moisture and a particular temperature; in some embodiments, a warm, moist environment is desirable. While not required, it may also be desirable to establish or maintain a sterile environment.

Additionally, it may be desirable to include other therapeutically beneficial agents in the formulation. For example, the vehicles or carriers may also include humectants or moisturizers to maintain a desired moisture level in the treated area. Other possibilities include drugs such as anesthetics or antibiotics, which provide other desired effects. Again, the possibilities are unlimited and are left to the practitioner.

The following Examples are provided to even more clearly describe and explain the invention.

### EXAMPLES

### Example 1

### Presence of ROS at the Wound-Site

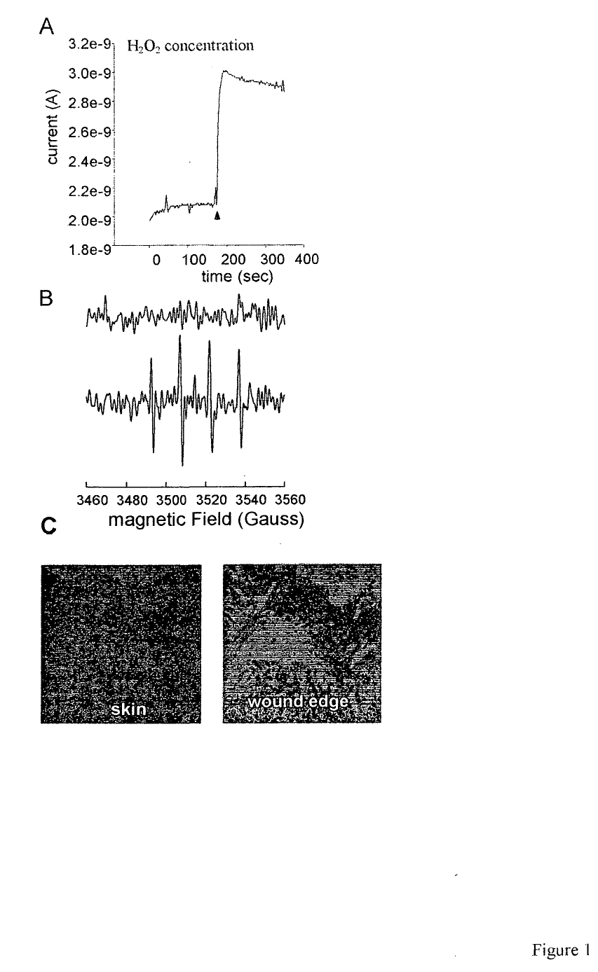

H₂O₂ concentration in wound fluid. Hunt/Schilling wire mesh cylinders were subcutaneously implanted on the back of 5-week-old C57BL/6 mice via incisional wounding. After five days, the wound fluid was collected and the steady-state H₂O₂ concentration in the fluid measured using a real-time electrochemical technique as described in Liu and Zweier (Free Radic Biol Med. Oct. 1, 2001;31(7):894-901). The baseline was collected in PBS. The results are shown in FIG. 1A.

Wound fluid (0.15 ml) was added to DPBS (1 ml) at time indicated with an arrow. Using a standard curve, the concentration of H₂O₂ determined in the wound fluid was 1.1 μM. This result is in contrast to measurements of blood plasma, which do not show measurable hydrogen peroxide.

H₂O₂ Detection at Wound Edge.

Hydrogen peroxide was tested at the wound edge by detection of radical footprints of hydrogen peroxide in the wound cavity using a spin-trap solution to rinse the exposed wound. Twelve hours after wounding, the site was treated with the spin trap, DMPO (5,5-dimethyl-pirrolin-1-oxyl). To obtain control data, a freshly inflicted wound was subjected to the same spin-trap treatment. After 15 minutes, the spin trap solution was withdrawn from the wound cavity and subjected to electron paramagnetic resonance (EPR) assay.

EPR measurements were performed using a Bruker ER 300 EPR spectrometer operating at X-band with a TM 110 cavity. FIG. 1B shows EPR spectra of DMPO adduct measured from wound rinsate. The spectra were acquired from DMPO (100 mM, 0.1 ml) effluents collected from wound cavity at 0 h (sham control, upper panel) 12 h post-wounding (lower panel). The spectra in the lower panel was identified as that of DMPO—OH with the following coupling constants: aN=14.90 G, aH=14.90. The data acquisition parameters were: microwave frequency, 9.8682 GHz; sweep width, 100 G; microwave power, 20 mW; modulation amplitude, 0.5 G; modulation frequency, 100 kHz; time constant 80 msec.

While the spectrum from the sham-treated spin trap solution did not shown any prominent spin adduct, the spectrum obtained from wound rinsate at 12-hour post-wound period showed a clear 1:2:2:1 quartet pattern. The individual components were identified as DMPO—OH (hydroxyl radical adduct) by simulation.

Superoxide production in normal skin and wound edge tissue. As a functional outcome of NADPH oxidase activity, O₂⁻ generation is often measured from tissue cryosections using dihydroethidium (DHE) as the ROS-sensitive fluorescent dye. Briefly, the wound edge samples were harvested at 12 hours after wounding and immediately frozen in OCT. Fresh 30-micron sections were incubated with DHE (0.01 mM, 20 min, 200x,) to detect O₂⁻ and visualized by confocal microscopy. Results are shown in FIG. 1C.

Employing this approach, it was clearly observed that the edge of wound-tissue stained much more prominently compared to freshly sliced normal skin. This finding lends further support that the wound site is enriched in ROS.

Together, these experiments clearly demonstrate that ROS, including hydrogen peroxide, are present in wound-healing tissue.

### Example 2

### Effect of Excess Catalase on Wound-Healing

Because hydrogen peroxide was shown to be present in wound-healing tissue, experiments were performed to determine whether decreasing its concentration would hinder wound healing. Catalase is the natural enzyme that hydrolyzes hydrogen peroxide, so it was introduced into wounds to be tested.

Briefly, catalase was introduced into wounds by its overexpression using an adenoviral vector. This vector allowed for high efficiency of over-expression in the murine skin. The skin to be wounded was subcutaneously injected once with either catalase or LacZ (control) adenoviruses (10¹¹ pfu) five days before wounding to allow for maximum over-expression of catalase at the wound site. Two 8×16 mm full-thickness secondary intention wounds were placed on the dorsal skin of eight-week-old C57BL/6 mice.

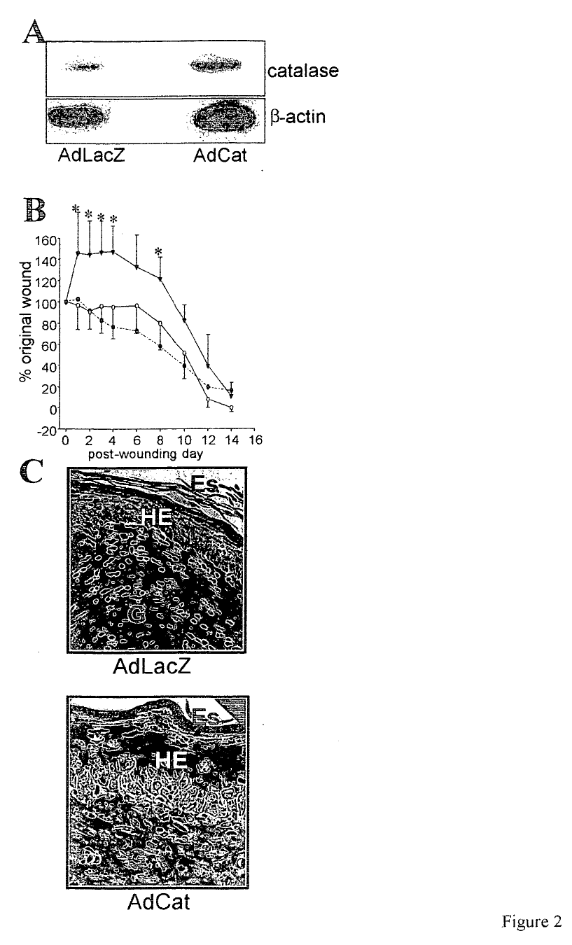

FIG. 2A shows a Western blot of infected skin showing catalase over-expression in the side treated with Ad-catalase (AdCat) virus compared to the side treated with Control Ad-LacZ virus. Blots were re-probed with β-actin to show equal loading of samples.

FIG. 2B shows wound closures as a percentage of area of initial wound determined on the indicated day after wounding. The dotted line represents a standard healing curve of saline treated C57BL/6 mice (open circles, ◯) without viral infection. AdCat Treatment (closed triangles,▾); AdlacZ treatment (closed circles,●); *p<0.05, compared to LacZ treated side.

FIG. 2C shows Masson trichrome staining performed on formalin-fixed paraffin sections of regenerated skin at the wound-site sampled on the day both wounds closed. The AdCat side shows broader HE region, indicative of incomplete (vs. control) regeneration of skin, consistent with slower closure. Es, eschar; G, granulation tissue; HE, hyperproliferative epithelium.

### Example 3

### Effect of Hydrogen Peroxide on Wound Closure

As it was clearly demonstrated that hydrogen peroxide is present in healing wounds (Example 1), and that its absence from healing wounds attenuates the healing process (Example 2), experiments were performed to examine the effect of added hydrogen peroxide.

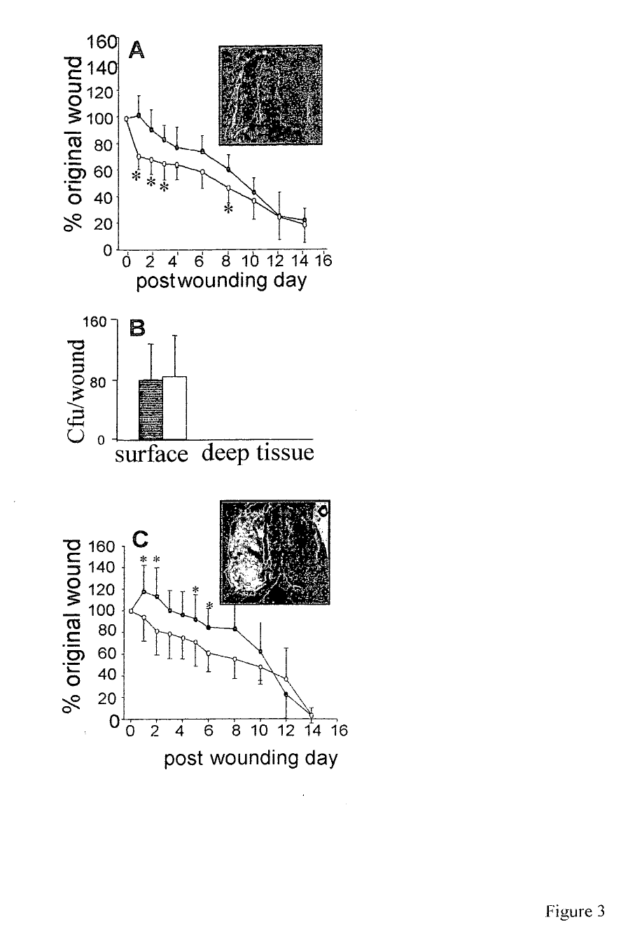

Briefly, two 8×16-mm full-thickness excisional wounds (FIG. 3, insets) were placed on the dorsal skin of C57BL/6 male mice (8 weeks old). Each of the two wounds was topically treated either with H₂O₂ or saline.

FIG. 3A shows low-dose of H₂O₂ (1.25 micromoles/wound; or 0.025 ml of 0.15% solution/wound; once daily, days 0-4, open circles, ◯) treatment facilitated closure moderately compared to placebo treated (closed circles,●) side. *, p<0.05.

FIG. 3B shows that low dose H₂O₂ treatment does not influence wound microflora. For determination of surface microflora, wounds (treated with either 1.25 micromoles H₂O₂/wound, open bar, or saline, closed bar) were swabbed (24-48 h post wounding) for 20 sec with an alginate-tipped applicator. Quantitative assessment of surface bacterial load was performed. For deep tissue wound microflora, 48 hours after wounding eschar tissue was removed, wound bed tissue underneath eschar was sampled, and quantitative assessment of bacterial load was performed. Values shown represent mean±SD of CFU of four observations.

FIG. 3C shows high dose (high, 25 micromoles/wound; closed circles, ●, 0.025 ml of 3% solution, versus low, 1.25 micromoles/wound or 0.025 ml of 0.15%; open circles, ◯, once daily days 0-4) of H₂O₂ adversely affected closure. (*, p<0.05; compared to low dose H₂O₂ treatment.) A higher concentration (inset) of H₂O₂ (62.5 micromoles/wound, left side treated; 0.025 ml of 7.5% solution/wound, once on day 0) treatment caused necrotic tissue damage and severe injury, leading to death of the mice.

### Example 4

### Wound and H₂O₂-Induced Changes in Angiogenesis Related Genes, Vascularization, and Wound-Edge Blood Flow

Additional experiments were performed to study the mechanism by which low doses of hydrogen peroxide increased the rate of wound healing.

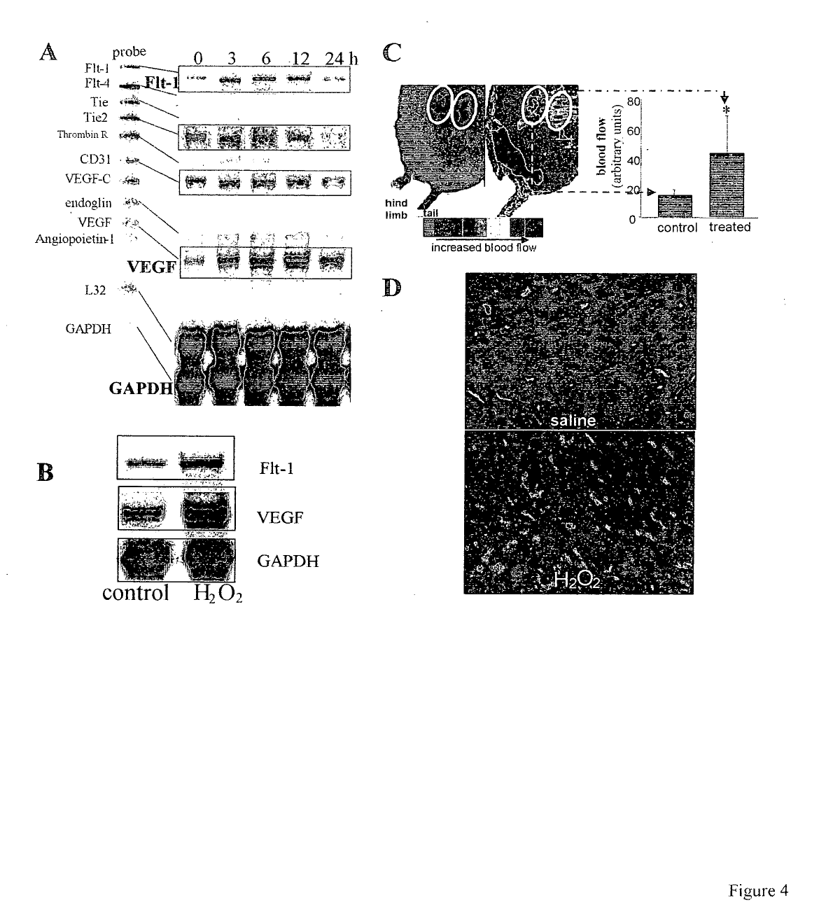

Paired excisional wounds were either treated with placebo saline or H₂O₂ (1.25 micromole/wound, days 0-4, once daily). Wound-edge tissue was collected at indicated times after wounding. FIG. 4A shows a ribonuclease protection assay (RPA) showing kinetics of angiogenesis-related mRNA expression in a placebo-treated wound. FIG. 4B shows how low-dose H₂O₂ treatment (1.25 micromole/wound, once daily, days 0-4) to wounds further augmented wound-induced Flt-1 and VEGF mRNA expressions as determined using RPA.

FIG. 4C shows blood-flow imaging of wounds performed non-invasively using a Laser Doppler blood-flow imaging device. Images reflecting the blood flow (right panel) and a digital photo (region of interest; left panel) from post-heal tissue are shown. Data of the blood flow is presented as mean±SD (bar graph). The mean represents the arithmetic mean of all valid blood flow values for pixels within the region of interest. The results show that the treatment resulted in increased blood flow, a functional outcome of enhanced angiogenesis.

FIG. 4D shows the results from day 8 post-wounding. Wound-edge was cryosectioned and vascularization was estimated by staining for CD31 (red, rhodamine) and DAPI (blue, nuclei); higher abundance of CD31 red stain in section obtained from H₂O₂ treated side (bottom) reflect better vascularization vs control (top).

### Example 5

### H₂O₂-Induced Phosphorylation of Focal Adhesion Kinase (FAK) in Microvascular Endothelial Cells and Wound-Edge Tissue

Human microvascular endothelial cells (HMEC-1) were treated with H₂O₂ for indicated dose and duration. Phosphorylation of FAK was detected using Western blot and phosphorylation site-specific antibodies against FAK. Native FAK or β-actin was blotted to show equal loading.

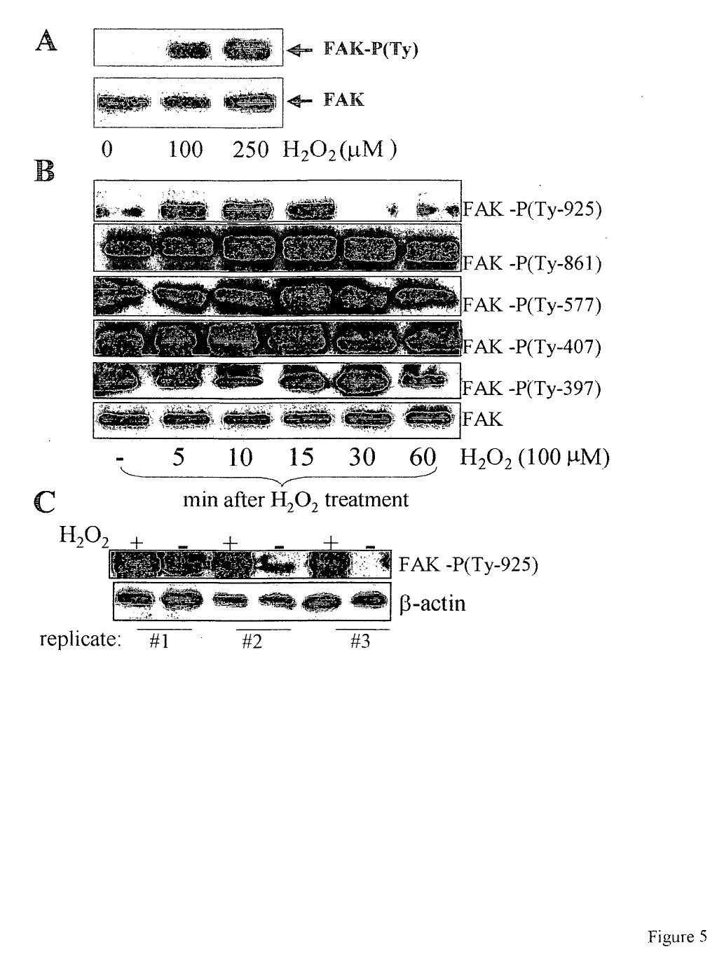

FIG. 5A shows the effect of various doses of H₂O₂ treatment on the phosphorylation (Ty 925) state of FAK. FIG. 5B shows the kinetics of site-specific activation phosphorylation of FAK in HMEC cells following H₂O₂ (0.1 mM) treatment.

In FIG. 5C, paired excisional wounds were either treated with placebo saline or H₂O₂ (1.25 micromole/wound). Wound-edge tissue was collected 30 minutes after wounding. FAK phosphorylation in wound edge tissue was determined using Western blot. Data from three animals (#1-#3) are shown.

### Example 6

### MCP-1 and p47phox Deficiency Impairs Dermal Healing

By attracting hydrogen-peroxide producing macrophages, monocyte/macrophage chemoattractant/chemotactic protein-1 (MCP-1) plays a key role in the development and resolution of the acute inflammatory response to wounding. p₄₇^{phox} is a regulatory subunit of NADPH oxidase, which is involved in ROS production. Because of the importance of these factors in ROS production and in wound healing, tests were performed to examine how hydrogen peroxide affects wounds in animals lacking these factors.

Briefly, two excisional wounds were placed on the dorsal skin of eight-week-old C57BL/6, MCP-1 or p47^{phox} knockout mice. Each of the two wounds was treated with either saline or H₂O₂ (1.25 micromoles/wound; days 0-4).

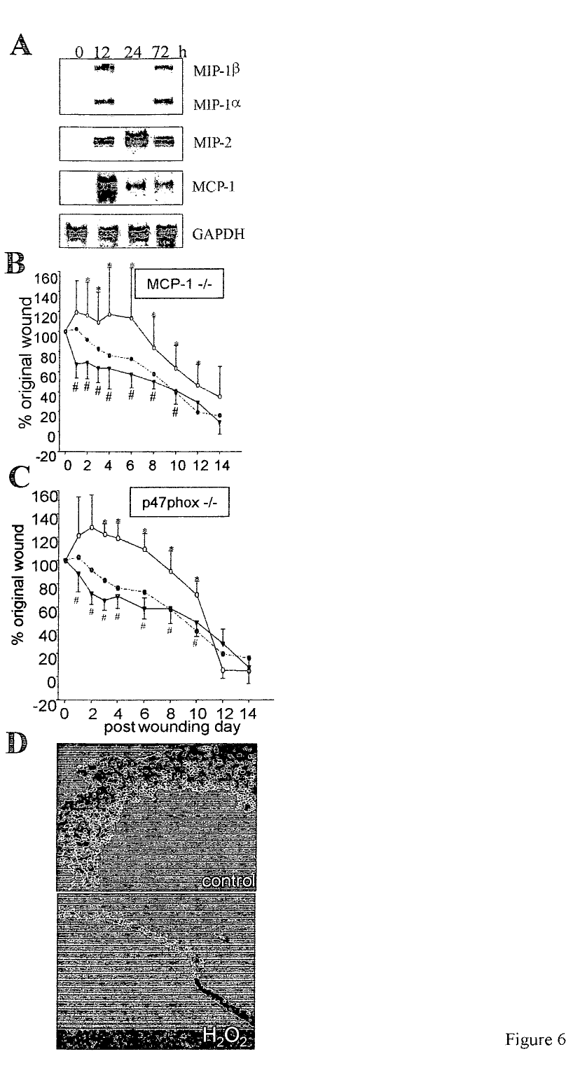

FIG. 6A shows an RNase protection assay showing the kinetics of monocyte/macrophage chemotactic protein related mRNA expression in placebo-treated wounds of wild-type (C57BL/6) mice. FIG. 6B shows wound closures in saline (closed circles, ●) treated wounds of C57BL/6, and H₂O₂ (closed triangles,▾) or saline (open circles, ◯) treated MCP-1 knockout mice are shown as percentage of area of initial wound. (* p<0.05; compared to C57BL/6 saline treatment. #, p<0.05; compared to knockout saline treatment.) FIG. 6C shows wound closures in saline (closed circles, ●) treated wounds of C57BL/6 and H₂O₂ (closed triangles,▾) or saline (open circles, ◯) or p47^{phox} knockout mice are shown as percentage of area of initial wound. *p<0.05; compared to C57BL/6 saline treatment. #, p<0.05; compared to KO saline treatment.

Keratin 14 supports epidermal differentiation and regeneration and its expression is triggered by dermal wounding. FIG. 6D shows keratin 14 (green fluorescence) expression in skin of p47^{Phox} knockout mice harvested from wound sites after closure on day 18 post-wounding. Note higher expression of keratin 14 in control side compared to H₂O₂-treated side indicating that in the control side healing is ongoing and incomplete , while H₂O₂-treated side shows that keratin 14 expression is comparable to normal skin indicating complete healing.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method of increasing the rate of lesion healing in mammals comprising applying to the lesion about 500 nanomoles to about 50 micromoles of hydrogen peroxide per square centimeter of lesion.

2. The method according to claim 1, comprising applying about 1 to about 50 micromoles of hydrogen peroxide per square centimeter of lesion.

3. The method according to claim 2, comprising applying about 1 to about 10 micromoles of hydrogen peroxide per square centimeter of lesion.

4. The method according to claim 3, comprising applying about 1 to about 2 micromoles of hydrogen peroxide per square centimeter of lesion.

5. The method according to claim 1, wherein the hydrogen peroxide applied to the lesion in a source chosen from enzymatic and chemical sources.

6. The method according to claim 5, wherein the source of hydrogen peroxide is chemical, and the source is hydrogen peroxide.

7. A method of increasing the rate of lesion healing in a mammal comprising applying hydrogen peroxide to the lesion at a rate of about 500 nanomoles to about 50 micromoles of hydrogen peroxide per square centimeter of lesion over a period of from about 12 hours to about 24 hours.

8. The method according to claim 7, wherein the hydrogen peroxide is applied at a rate of from about 1 to about 10 micromoles.

9. The method according to claim 7, wherein the hydrogen peroxide is applied in a pharmaceutically acceptable composition.

10. The method according to claim 9, wherein the pharmaceutically acceptable composition is in a form chosen from gels, lotions, ointments, creams, pastes, and liquids.

11. The method according to claim 7, wherein the hydrogen peroxide is applied in a pharmaceutically acceptable device.

12. The method according to claim 11, wherein the pharmaceutically acceptable device is chosen from bandages, surgical dressings, gauzes, adhesive strips, surgical staples, clips, hemostats, intrauterine devices, sutures, trocars, catheters, tubes, and implants.

13. The method according to claim 12, wherein the implant is chosen from pills, pellets, rods, wafers, discs, and tablets.

14. The method according to claim 11, wherein the device comprises a polymeric material.

15. The method according to claim 14, wherein the polymeric material comprises an absorbable material.

16. The method according to claim 15, wherein the absorbable material comprises a synthetic material.

17. The method according to claim 16, wherein the synthetic material is chosen from cellulosic polymers, glycolic acid polymers, methacrylate polymers, ethylene vinyl acetate polymers, ethylene vinyl alcohol copolymers, polycaptrolactam, polyacetate, copolymers of lactide and glycolide, polydioxanone, polyglactin, poliglecaprone, polyglyconate, polygluconate, and combinations thereof.

18. The method according to claim 15, wherein said absorbable material comprises a non-synthetic material.

19. The method according to claim 18, wherein said non-synthetic material is chosen from catgut, cargile membrane, fascia lata, gelatin, collagen, and combinations thereof.

20. The method according to claim 14, wherein the polymeric material comprises a nonabsorbable material.

21. The method according to claim 20, wherein the nonabsorbable material comprises a synthetic material.

22. The method according to claim 21, wherein the synthetic material is chosen from nylons, rayons, polyesters, polyolefins, and combinations thereof.

23. The method according to claim 20, wherein the nonabsorbable material comprises a non-synthetic material.

24. The method according to claim 23, wherein said non-synthetic material is chosen from silk, dermal silk, cotton, linen, and combinations thereof.

25. The method according to claim 7, wherein the lesion is chosen from wounds, ulcers, and burns.

26. The method according to claim 25, wherein the wounds are chosen from acute wounds and chronic wounds.

27. The method according to claim 25, wherein the wounds are chosen from full thickness wounds and partial thickness wounds.

28. The method according to claim 26, wherein the acute wounds are chosen from surgical wounds, penetrating wounds, avulsion injury, crushing injury, shearing injury, burn injury, laceration, and bite wound.

29. The method according to claim 26, wherein the chronic wounds are chosen from arterial ulcers, venous ulcers, pressure ulcers, and diabetic ulcers.

30. A hydrogen peroxide delivery device for administration to a lesion, comprising hydrogen peroxide and a carrier material, which device releases said hydrogen peroxide for a period of time which is at least about 12 hours, wherein the hydrogen peroxide released from the device is in insufficient concentration to produce necrosis of the lesion.

31. The hydrogen peroxide delivery device according to claim 30, wherein the device releases from about 0.5 to 50 μmol hydrogen peroxide/cm² wound/12 hr to about 0.5 to 50 μmol hydrogen peroxide/cm² wound/24 hr.

32. The hydrogen peroxide delivery device according to claim 31, wherein the carrier material comprises a polymeric material.

33. The hydrogen peroxide delivery device according to claim 32, wherein the polymeric material comprises an absorbable material.

34. The hydrogen peroxide delivery device according to claim 32, wherein the polymeric material comprises a synthetic material.

35. A composition for treating lesions in mammals comprising: hydrogen peroxide and a pharmaceutically acceptable carrier, wherein a unit dose of the composition comprises from about 0.5 to about 50 μmol hydrogen peroxide/cm² wound.

36. The composition according to claim 35, wherein the carrier comprises a gel material.

37. The composition according to claim 35, wherein the carrier comprises a liquid material.
